(19) United States
(12) Patent Application Publication
(10) Pub. No.: US 2003/0093048 A1
(43) Pub. Date: May 15, 2003

(54) Acquisition distribution layer having void volumes for an absorbent article

(76) Inventor: Robert K. McBride, Jasonville, IN (US)

Correspondence Address: JENKENS & GILCHRIST, PC, 1445 ROSS AVENUE, SUITE 3200, DALLAS, TX, 75202, US

(21) Appl. No.: 10/228,926

(22) Filed: Aug. 26, 2002

Related U.S. Application Data
(63) Continuation-in-part of application No. 09/668,649, filed on Sep. 22, 2000.

(51) Int. Cl. A61F 13/15

(52) U.S. Cl. 604/368; 604/378; 604/374

(57) ABSTRACT

The invention relates to an absorbent article having a topsheet and an absorbent core material. An acquisition distribution layer (ADL) is located between the topsheet and the absorbent core material. By employing a high void volume acquisition distribution layer the absorbent article can achieve greater strikethrough and rewet characteristics without altering the core design, or the absorbent article may achieve current design criteria with less absorbent core material or less expensive absorbent core material. A method is disclosed for designing an absorbent article to reduce the bulk of the absorbent core. Furthermore, a method is disclosed for reducing the cost of an absorbent article by using a high void volume ADL along with less expensive core materials, while maintaining the strikethrough and rewet performance of the absorbent article.

This application is a continuation-in-part of co-pending U.S. application Ser. No. 09/668649 filed on Sep. 22, 2000.

### FIELD OF THE INVENTION

This invention relates to absorbent articles such as diapers, incontinent articles, sanitary napkins, and the like. More particularly, this invention relates to absorbent articles having a topsheet and a film acquisition distribution layer having a void volume space.

### BACKGROUND OF THE INVENTION

A variety of absorbent articles that are adapted to absorb body fluids are well known. Examples of absorbent articles include diapers, incontinent articles, and sanitary napkins.

One problem associated with known absorbent articles is waste product leakage, which may contaminate clothing articles, such as pants, shirts, and bedding. The amount of leakage experienced by a wearer can be reduced by increasing the rate that liquid enters the absorbent core Therefore, an absorbent article wherein liquid rapidly penetrates the topsheet and is contained in the absorbent core will experience less leakage than an absorbent article wherein liquid is able to run across the topsheet before penetrating into the absorbent core. Consequently, run-off reduction reduces the amount of leakage associated with an absorbent article

Another problem associated with absorbent articles is dryness of the skin contacting surface of the article. Generally, the drier the skin contacting surface, the more comfortable the absorbent article. Attempts have been made to reduce surface wetness in disposable diaper structures. For example, U.S Pat. No. 3,945,386 issued to Anczurowski on Mar. 23, 1976 and U.S. Pat. Nos. 3,965,906 and 3,994,299 issued to Karami on Jun. 29, 1976 and Nov. 30, 1976, respectively, teach diaper structures having a perforated thermoplastic film interposed between the topsheet and the absorbent core U.S. Pat. No 4,324,247 issued to Aziz on Apr. 13, 1982 describes an effort directed to both reducing run-off and reducing the surface wetness of absorbent articles.

In addition to the dryness of the skin contacting surface, the feet of the skin contacting surface is also an important consideration One problem is that some consumers do not like the plastic feel associated with formed films. A number of efforts have been directed at improving the feel of the surface of absorbent articles. One example is described in U.S. Pat. No. 3,967,623 issued to Butterworth, et al. The Butterworth patent teaches an absorbent pad having a facing sheet made of a perforated thermoplastic web that has an integral fibrous or sueded outer surface.

An additional problem with typical absorbent articles, in particular adult incontinence diapers. As a wearer urinates a second time or more, a sensation of wetness is felt as unabsorbed fluid flows laterally through the topsheet from an area of saturated core material to an area of unsaturated core material for absorption. This sensation is highly uncomfortable and undesirable.

A further problem with a typical absorbent article is the overall thickness of the absorbent article. In conforming to the human body, an absorbent article may tend to bunch and bulge if it is too thick In a typical absorbent article the absorbent core is the thickest part. It, therefore, would be advantageous to use a thinner core. The traditional tradeoff when a thinner core is used is a critical increase in strikethrough times and rewet measures. In other words, while thinner cores are known to the art, heretofore it has been necessary to balance the discomfort of a thick absorbent core against the discomfort of prolonged strikethrough and increased rewet. Therefore, a design criteria is typically specified wherein an acceptable level of strikethrough and an acceptable level of rewet are predetermined and the least amount of absorbent core material is used to achieve the design criteria. It would be very advantageous to have an absorbent article which could achieve these design criteria with less core material, or in the alternative, achieve even better performance with the existing core material.

Another problem with a typical absorbent article, is that heretofore, the only means for increasing the performance of the absorbent core was to use either more material or a more expensive material. To that end, a mixture of relatively cheap pulp material is typically employed with the addition of a more expensive super absorbent polymer (SAP). While the SAP significantly increases the performance of the pulp absorbent core, there is a limit to how much SAP may be used before the expanding SAP gelblocks (chokes the flow of fluid through the core) with an accompanying loss of performance (2nd & 3rd strikethroughs and rewets are higher). Therefore, it would be advantageous to design an absorbent core with less SAP in order to save costs without reducing either strikethrough or rewet performance of the absorbent article. Furthermore, it would be advantageous to design an absorbent article which requires less SAP and less pulp without reducing either strikethrough or rewet performance.

The products described in most of the above references, however, are less than ideal in achieving a good combination of all three desired properties, reduced surface run-off, improved ability to prevent a feeling of wetness of the topsheet, and improved feel, with the freedom to design a less bulky absorbent article or a cheaper absorbent article.

### SUMMARY OF THE INVENTION

The invention relates to an absorbent article having a topsheet and an absorbent core material. An acquisition distribution layer (ADL) is located between the topsheet and the absorbent core material. By employing a high void volume acquisition distribution layer the absorbent article can achieve improved strikethrough and rewet characteristics without altering the core design, or the absorbent article may achieve current design criteria with less absorbent core material or less expensive SAP in the absorbent core material. A method is disclosed for designing an absorbent article to reduce the bulk of the absorbent core. Furthermore, a method is disclosed for reducing the cost of an absorbent article by using a high void volume ADL along with less expensive core materials, while maintaining the strikethrough and rewet performance of the absorbent article.

### BRIEF DESCRIPTION OF THE DRAWINGS

FIG. 1 is perspective view of an absorbent article of the invention that utilizes an acquisition distribution layer.

FIG. 2 is a cross sectional schematic view of the absorbent article of FIG. 1 taken along line 2-2 wherein the acquisition distribution layer is of a prior art type.

FIG. 3 is an enlarged cross sectional view of the prior art acquisition distribution layer of FIG. 2.

FIG. 4 is a plan view of a three dimensional apertured film used in a first embodiment of the invention for use as an acquisition distribution layer in the absorbent article of FIG. 1

FIG. 5 is a cross sectional view of the absorbent article of FIG. 1 taken along line 2-2 of FIG. 1 wherein the acquisition distribution layer shown is a cross sectional view of the three dimensional apertured film of FIG. 4 taken along line 5-5 of FIG. 4.

FIG. 6 is a plan view of a three dimensional apertured film used in a second embodiment of the invention for use as an acquisition distribution layer in the absorbent article of FIG. 1

FIG. 7 is a cross sectional view of the absorbent article of FIG. 1 taken along line 2-2 of FIG. 1 wherein the acquisition distribution layer shown is a cross sectional view of the three dimensional layer apertured film of FIG. 6 taken along line 7-7 of FIG. 6.

FIG. 8 is a plan view of a three dimensional apertured film used in a third embodiment of the invention for use as an acquisition distribution layer in the absorbent article of FIG. 1.

FIG. 9 is a cross sectional view of the absorbent article of FIG. 1 taken along line 2-2 of FIG. 1 wherein the acquisition distribution layer shown is a cross sectional view of the three dimensional layer apertured film of FIG. 8 taken along line 9-9 of FIG. 1.

FIG. 10 is a plan view of a disposable diaper utilizing the three dimensional apertured film of FIGS. 8 and 9.

FIG. 11 is a cross sectional view of the absorbent article of FIG. 1 wherein the acquisition distribution layer is a multi-layer apertured film used in a fourth embodiment of the invention.

FIG. 12 is a cross sectional view of the absorbent article of FIG. 1 wherein the acquisition distribution layer is a multi-layer apertured film used in a fifth embodiment of the invention.

FIG. 13 is a schematic drawing an Liquid Acquisition Apparatus that is used to test the various embodiments of the absorbent articles of FIGS. 1-12.

FIG. 14 is a graphical representation of data from Table 1 that shows Total Fluid Overflow and Inverse Loft for various samples of absorbent articles shown in FIGS. 1-12

FIG. 15 is a plan view at 50× magnification of Sample 2 and Sample 4 for purposes of comparing the void volume space of the samples.

FIG. 16 is a cross-sectional view at 50× magnification of Sample 2 and Sample 4 for purposes of comparing the void volume space of the samples.

### DETAILED DESCRIPTION OF THE INVENTION

This invention relates to absorbent articles having a three dimensional apertured film acquisition distribution layer. Examples of absorbent articles include diapers, incontinent articles, sanitary napkins, and similar articles.

Definitions

For purposes of this application, the term “absorbent article” will refer to articles that absorb and contain body exudates. More specifically, the term refers to articles which are placed against or in proximity to the body of a wearer for absorbing and containing various exudates discharged from the body. The term “absorbent article”, as used herein, is intended to include diapers, incontinent articles, sanitary napkins, pantiliners, and other articles used to absorb body exudates.

The term “diaper” refers to a garment typically worn by infants and incontinent persons that is drawn up between the legs and fastened about the waist of the wearer. Examples of diapers from the prior art include diapers described in U.S. Pat Re. No. 26,152, issued to Duncan, et al. on Jan. 31, 1967, U.S. Pat No. 3,860,003 issued to Buell on Jan. 14, 1975; U.S. Pat. No 4,610,678 issued to Weisman, et al on Sep. 9, 1986; U.S. Pat No. 4,673,402 issued to Weisman, et al on Jun. 16, 1987; U.S. Pat No. 4,695,278 issued to Lawson on Sep. 22, 1987, U.S. Pat. No. 4,704,115 issued to Buell on Nov. 3, 1987; U.S. Pat. No. 4,834,735 issued to Alemany, et al. on May 30, 1989; U.S. Pat. No. 4,888,231 issued to Angstadt on Dec. 19, 1989; and U.S. Pat. No. 4,909,803 issued to Aziz, et al. on Mar. 20, 1990.

The term “incontinent article” refers to pads, undergarments, e.g., pads held in place by a suspension system, such as a belt, or other device, inserts for absorbent articles, capacity boosters for absorbent articles, briefs, bed pads, and similar devices, whether worn by adults or other incontinent persons. Examples of incontinent articles include those disclosed in U.S. Pat. No. 4,253,461 issued to Strickland, et al. on Mar. 3, 1981; U.S. Pat. Nos. 4,597,760 and 4,597,761 issued to Buell; the above-mentioned U.S. Pat. Nos. 4,704,115; 4,909,802 issued to Ahr, et al.; U.S. Pat. No 4,964,860 issued to Gipson, et al. on Oct. 23, 1990, and in U.S patent Application Ser. Nos. 07/637,090 and 07/637,571 filed respectively by Noel, et al. and Feist, et al. on Jan. 3, 1991.

The term “sanitary napkin” refers to an article that is worn by a female adjacent to the pudendal region that is intended to absorb and contain various exudates which are discharged from the body, e.g., blood, menses, and urine. Examples of sanitary napkins are disclosed in U.S. Pat. No. 4,285,343, issued to McNair on Aug. 25, 1981; U.S. Pat. Nos. 4,589,876 and 4,687,478, issued to Van Tilburg on May 20, 1986 and Aug. 18, 1987 respectively; U.S. Pat. Nos. 4,917,697 and 5,007,906 issued to Osborn, et al on Apr. 17, 1990 and Apr. 16, 1991, respectively, and U.S. Pat. Nos. 4,950,264, and 5,009,653 issued to Osborn on Aug. 21, 1990 and Apr 23, 1991, respectively; and in U.S. patent application Ser. No. 07/605,583 filed Oct. 29, 1990 in the name of Visscher, et al.

The term “pantiliner” refers to absorbent articles that are less bulky than sanitary napkins that are generally worn by women between their menstrual periods. Examples of pantiliners are disclosed in U.S. Pat. No. 4,738,676 entitled “Pantiliner” issued to Osborn on Apr. 19, 1988.

The term “finished absorbent article” is used herein to generally mean any absorbent article having incorporated all layers of material and other features that the article is intended to have which affect the product's performance characteristics during its intended use. This term includes, but is not limited to, products well known in the art as diapers, sanitary napkins, and adult incontinent briefs.

The term “finished product testing” is used herein to refer to the testing of finished absorbent articles which results in the generation of data relating to the performance of a finished absorbent article or other characteristics of a finished absorbent article.

The term “insult” is used herein to refer to the act of applying a finite amount of liquid to the topsheet of a finished absorbent article. An insult may occur during product use and during finished product testing. Consequently, “multiple insults” occur when the same finished absorbent article is insulted more than once. Multiple insults may occur during product use and during finished product testing.

The term “topsheet” is used herein to refer to the layer of material in a finished absorbent article which is first contacted by liquid during an insult when the article is properly used It is well known in the art that many finished absorbent articles employ thin sheets of nonwoven materials or perforated films as a topsheet. However, this definition of topsheet is not limited to mean only sheets of nonwoven layers and perforated films, but instead includes any material composition and in any shape, form, or structure which is the layer first contacted by liquid during an insult when the article is properly used.

The term “finished product test” is used herein to refer to the test which results in the generation of data which identifies a finished absorbent article's finished product liquid strike-through time (absorbency rate or liquid acquisition rate) and finished product wetback (rewet)

Absorbent Articles

The disclosures of all patents, patent applications and any patents which issue therefrom, as well as any corresponding published foreign patent applications, and publications mentioned throughout this patent application are hereby incorporated by reference herein. It is expressly not admitted, however, that any of the documents incorporated by reference herein teach or disclose the present invention. It is also expressly not admitted that any of the commercially available materials or products described herein teach or disclose the present invention.

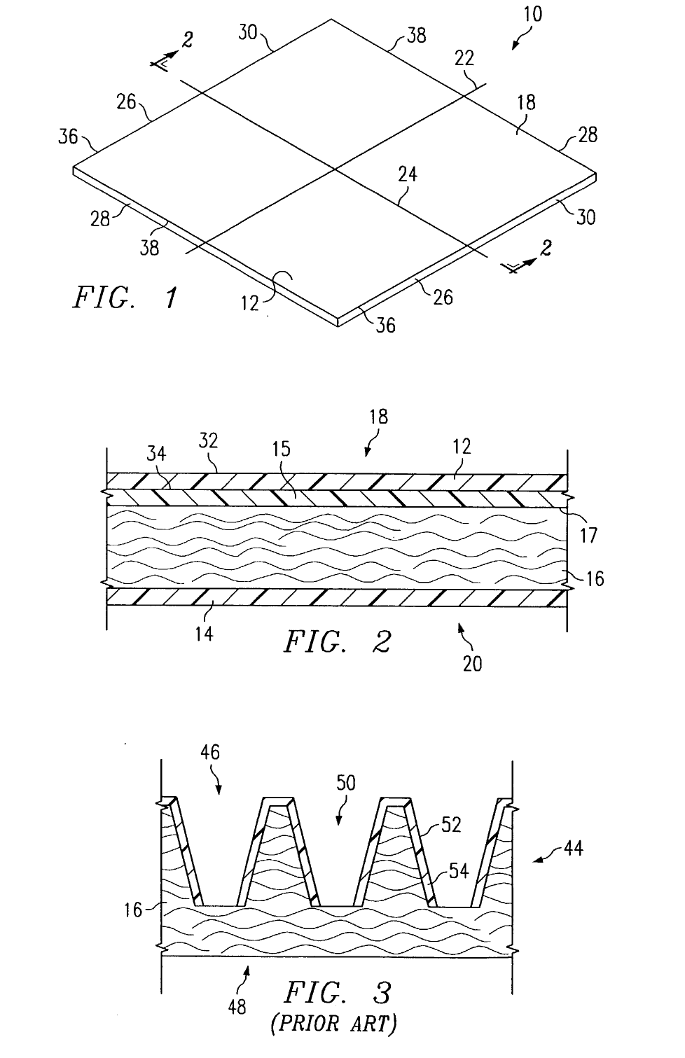

Referring now to FIG. 1, a simplified representation of a typical absorbent article 10 is shown. It should be understood, however, that FIG. 1 is shown for purposes of example only, and should not be construed to limit the particular type or configuration of absorbent article. As shown in FIG. 2, absorbent article 10 basically comprises topsheet 12, backsheet 14, an acquisition distribution layer 15, and an absorbent core 16 Absorbent core 16 has a top or body facing side 17.

The absorbent article 10 has two surfaces, a body-contacting surface or body surface 18 and a garment-contacting surface or garment surface 20. The body surface 18 is intended to be worn adjacent to the body of the wearer. The garment surface 20 (FIG. 2) of the absorbent article 10 is on the opposite side and is intended to be placed adjacent to the wearer's undergarments or clothing when the absorbent article 10 is worn.

The absorbent article 10 has two centerlines, a longitudinal centerline 22 (FIG. 1) and a transverse centerline 24 (FIG. 1). Absorbent article 10 has two spaced apart longitudinal edges 26 and two spaced apart transverse or end edges, i.e., ends 28, which together form the periphery 30 of the absorbent article 10.

The individual components of the absorbent article 10 will now be looked at in greater detail. Topsheet 12 is compliant, soft-feeling and non-irritating to the wearer's skin. Further, topsheet 12 is liquid permeable, permitting liquids to readily penetrate through its thickness. The topsheet 12 has a body-facing side 32 (FIG. 2) and a garment-facing side 34 (FIG. 2), two longitudinal or side edges 36 and two end edges 38 (FIG. 1). Absorbent core 16 has a top or body facing side 17. Throughout the remainder of this application, similar components will share the same numbers for all embodiments of the invention, e.g., “topsheet” will be designated by the numeral 12 in each embodiment.

Topsheet 12 is preferably made of a nonwoven material or of a vacuum formed film layer. Topsheet 12 may be bonded to acquisition distribution layer 15 (FIG. 2), although in a preferred embodiment, topsheet 12 is not bonded to but instead lays in contact with acquisition distribution layer 15. The absorbent article of FIG. 3 utilizes a three dimensional apertured plastic film 44 as an anti-rewet (or anti-wicking) layer. Three dimensional apertured plastic film 44 has a body facing side or female side 46 and a garment facing side or male side 48. The garment-facing side 34 of the topsheet 12 is preferably maintained in close contact with the female side 46 of the apertured plastic film 44. The topsheet 12 and acquisition distribution layer 15 are examined in greater detail below.

Where topsheet 12 is a nonwoven material, it may be any nonwoven fabric that is permeable to liquids. A suitable nonwoven fabric may be manufactured from a various materials including natural fibers (e.g., wood or cotton fibers), synthetic fibers (e.g., polyester, polypropylene) or a combination thereof. The topsheet 12 is preferably made from fibers selected from a group consisting of polypropylene, polyester, polyethylene, polyvinylalcohol, starch base resins, polyurethanes, cellulose and cellulose esters.

Various manufacturing techniques may be used to manufacture nonwoven fabric for use in topsheet 12. For example, the nonwoven fabric may be resin-bonded, needle punched, spunbonded, or carded. Carded nonwoven fabrics may be thermally bonded, air-thru bonded, and spunlaced fabrics. A preferred nonwoven fabric is a thermally bonded polypropylene fabric.

A typical nonwoven topsheet 12 is a nonwoven fabric having a pattern of thermal bond sites. One example of a nonwoven fabric has a carded thermally dot bonded polypropylene web. The thermal bonds of such a fabric are typically rectangularly-shaped in plan view. The bonds are typically arranged in staggered rows. Another typical nonwoven is a spunbonded polypropylene web with similarly arranged thermal bonds. Still another typical nonwoven fabric is a carded polypropylene web that is embossed in accordance with the method taught in U.S. Pat. No. 4,781,710 issued to Megison, et al. This nonwoven fabric has embossed and thermal bonded areas that are diamond-shaped in plan view. The diamond-shaped bonds are spaced apart and arranged in a diamond-shaped grid such as is shown in FIGS. 1 and 2 of the Megison, et al. patent. Typically, the embossing does not extend to the underlying core, however.

Where topsheet 12 is a vacuum formed film it may be any vacuum formed film that is apertured to allow passage of liquids. Such films may be made of several types of materials, including a variety of polymers, for instance, polyolefins and their copolymers and blends. Such films as those described in Raley U.S. Pat. Nos. 4,317,792, Mullane 4,324,246, Thomas 4,351,784, Thomas 4,456,570, and/or Radel 4,342,314 are further examples.

Core 16 may include materials manufactured by methods such as air laid, needle punched, hydroentangled, curly fiber, core fluff, air laid nonwoven fiber, pulp core fluff, etc. The core could be made from various materials such as polyolefins and their copolymers and blends, as well as cellulose and its blends with other thermoplastic polymers. Absorbent polymers in various forms may also be included in the core composition. The core or the absorbent layers may include nonwoven materials made from such natural and synthetic polymers as noted above, and may or may not include super absorbent polymers in the form of fibers, granules or powders. The most common, and economical, material is a blend of natural fiber pulp and super absorbent polymer granules or powders. Of these two materials, the super absorbent polymer is significantly more expensive.

Preferably, acquisition distribution layer (herein also referred to as “ADL”) 15 is a perforated thermoplastic film with tapered capillaries which has a run off percent of less than about 10 percent and which has an increased liquid flow rate through the tapered capillaries. The method of making such a film includes a two-fold surface treatment, which is taught by U.S. Pat. Nos. 4,535,020 and 4,456,570 to Thomas et al entitled, “Perforated Film” and “Treatment of Perforated Film”, respectively. U.S. Pat. Nos. 4,535,020 and 4,456,570 are incorporated herein by reference. The method teaches that one surface treatment is provided by adding an internal chemical additive, namely a surfactant, to a film forming polyolefin resin. The additive is compounded or otherwise mixed or blended with the resin prior to the film being formed from the resin. After the film is formed the other surface treatment is accomplished by treating the film with a corona discharge treatment which acts on the chemical additive to provide the perforated film with a zero or near zero percent run off.

The surfactant provides a film surface which has greater polarizability than the polyolefin film would have without the surfactant being added. Higher surface polarity yields higher wettability. Although the chemically treated film is more polar than untreated film, corona discharge treatment of the film itself provides the desired maximum wettability. Any surfactant which achieves this polarity and which migrates to the surface of the film may be used in this invention.

Referring now to FIG. 3, the apertured plastic film 44 is typically located between the topsheet 12 and the absorbent core 16. As shown in FIG. 3, the apertured plastic film 44 is a three-dimensional structure having a plurality of tapered capillaries 50, each of which has a base opening 52, and an apex opening 54. The apex of the openings 54 are in intimate contact with the absorbent core 16 Additionally, most of the surface area of male side 48 of film 44 is in contact with core 16 while female side 46 is opposite core 16.

The apertured plastic film 44 is typically manufactured from a liquid impervious, thermoplastic material. One suitable material is a low density polyethylene film having a thickness of from 0.001 to 0.002 inches (0.0025 to 0.0051 cm.). The thermoplastic material for use in the manufacture of a typical apertured plastic film 44 is selected from a group consisting generally of polyethylene, polypropylene, polyvinyl chloride, starch base resins, polyvinylalcohol, polyurethanes, polycaprolactone and cellulose esters, or combinations thereof.

In one typical embodiment, the thermoplastic material is provided with a multiplicity of tapered capillaries 50 in a manner, size, configuration, and orientation set forth in U.S. Pat. No. 3,939,135 issued to Thompson on Dec. 30, 1975. Other typical apertured plastic films are disclosed in U.S. Pat. No. 4,324,246, issued to Mullane, et al on Apr. 13, 1982, U.S. Pat. No. 4,342,314, issued to Radel, et al. on Aug. 3, 1982, and U.S. Pat. No. 4,463,045, issued to Ahr, et al. on Jul. 31, 1984. The apertured plastic film 44 can also consist of other types of apertured plastic films that are not thermoplastic. The type of film used depends on the type of processing that the film and nonwoven components are subjected to during the manufacture of the topsheet 12. Thermoplastic films are typically used when the topsheet 12 and the acquisition distribution layer 15 or film 44 are integrally formed into a composite structure by melting. Other types of apertured films include, but are not limited to hydro-formed films. Hydro-formed films are described in at least some of the following U.S. Pat. Nos.: 4,609,518, 4,629,643, 4,695,422, 4,772,444, 4,778,644, and 4,839,216 issued to Curro, et al., and U.S. Pat. No. 4,637,819 issued to Ouellette, et al.

Typically, the nonwoven fabric of topsheet 12 and the apertured plastic film 44 are placed into a face-to-face relationship. The two components may be secured or unsecured. The two components, if secured, may be secured to each other by various methods. Typical methods for securing the nonwoven fabric and the apertured film 44 include, but are not limited to adhesives, fusion including heat bonding and/or pressure bonding, ultrasonics, and dynamic mechanical bonding.

The adhesives can be applied in a uniform continuous layer, a patterned layer, or an array of separate lines, spirals, beads, or spots of adhesive. The adhesive attachment typically comprises an open pattern network of filaments of adhesive such as is disclosed in U.S. Pat. No. 4,573,986 issued to Minetola, et al on Mar. 4, 1986, or an open pattern network of filaments having several lines of adhesive filaments swirled into a spiral pattern as illustrated by the apparatus and method shown in U.S. Pat. No. 3,911,173 issued to Sprague, Jr. on Oct. 7, 1975; U S. Pat. No. 4,785,996 issued to Zieker, et al. on Nov. 22, 1978; and U.S. Pat. No 4,842,666 issued to Werenicz on Jun. 27, 1989. Another method of heat/pressure bonding that could be used is described in U.S. Pat. No 4,854,984 issued to Ball, et al. on Aug. 8, 1989.

The nonwoven, fabric of topsheet 12 and the apertured plastic film 44 may alternatively be indirectly secured. For example, the nonwoven fabric and the apertured film 44 could be secured to or through a thin layer of airfelt, or a layer of hydrophobic material positioned between the nonwoven fabric and the apertured plastic film 44. Typically, such additional layer or layers are treated with a surfactant as described in greater detail below.

The nonwoven fabric of topsheet 12 and the apertured plastic film 44 can alternatively be integrally formed into a composite structure, as taught by Merz et al. in U.S. Pat. No. 4,995,930. The terms “composite”, “composite structure” or “combination”, as used herein, refer to relationships in which portions of the nonwoven fabric extend into the film 44, and vice versa so that they are integrally attached.

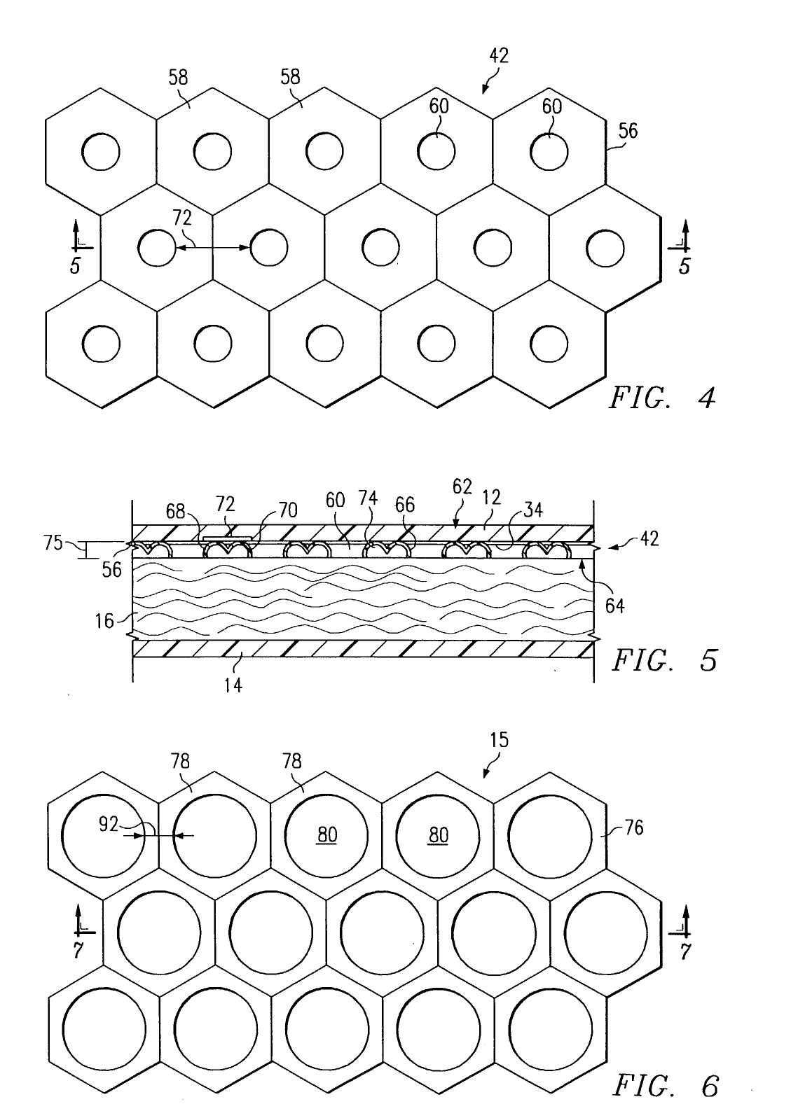

Referring now to FIGS. 4 and 5, a first embodiment of an improved absorbent article of the applicant's invention utilizes an acquisition distribution layer 42 made of a three dimensional apertured film 56 imparted with a hexagonal pattern. Although a hexagonal pattern is used for purposes of illustration, it should be understood that other patterns may also be used for any of the films described herein. Examples of other patterns include circular, oval, elliptical, polygonal, or other suitable patterns or combinations of patterns. The hexagonal pattern forms a plurality of adjacent hexagons or cells 58. In the preferred embodiment, the hexagonal pattern is based on a 8.75 mesh wherein “mesh” is the number of cells 58 aligned in a one-inch length. Although a mesh count of 8.75 is preferred, a mesh count of from 2 to 25 or more preferably from 4 to 15 may be used. Preferably, each cell 58 is provided with an aperture 60 that has a large hole diameter, e.g., 59 mils, which are large enough to allow insult fluids to be acquired through the three dimensional apertured film 56 as rapidly as the fluids are delivered.

Referring in particular to FIG. 5, which shows an enlarged cross sectional view of film 56 taken along line 5-5 of FIG. 4, three dimensional apertured film 56 has a body facing side or female side 62 and a garment facing side or male side 64. The garment-facing side 34 of the topsheet 12 is preferably maintained in close contact with the female side 62 of the apertured plastic film 56. Preferably topsheet 12 maintains in contact with film 56 but is unbonded to film 56.

As can be seen in FIG. 5, the film 56 is located between a topsheet 12 and an absorbent core 16. The apertured plastic film 56 is a three-dimensional structure having a plurality of capillaries 66, each of which has a base opening 68 and an apex opening 70. The apex openings 70 of the capillaries 66 are in intimate contact with the absorbent core 16, and preferably apex openings 70 are affixed to core 16 to insure this intimate contact. It should also be noted that essentially only the apex openings 70 of the capillaries 66 are in intimate contact with the core 16, thereby assuring that the void spaces 74 providing for lateral spillage remain substantially unencumbered. A land area 72 is formed between adjacent apertures 60 on the female side 62 of the apertured plastic film 56. A void volume space 74 (FIG. 5) is formed on the male side 64 of the apertured plastic film 56 that provides a fluid passageway between each of the cells 58. Preferably, the ratio of void volume space 74 versus apex opening space 70 is 2:1. The three dimensional apertured film 56 has a loft 75, i.e. the distance between the surface on the female side 62 and the planar surface on the male side 64, of from 0.031″ to 0.125″, more preferably 0.045″ to 0.100″, and most preferably of 0.050″. The thermoplastic material used in the film 56 preferably has a density in the range of from about 0.919 g/cc to 0.960 g/cc, with the more preferred range of densities being from about 0.930 g/cc to 0.950 g/cc. The general melt indices range for a typical material is preferably from about 0.10 to about 8.50, with the more preferred range typically being from about 1.5 to about 4.5.

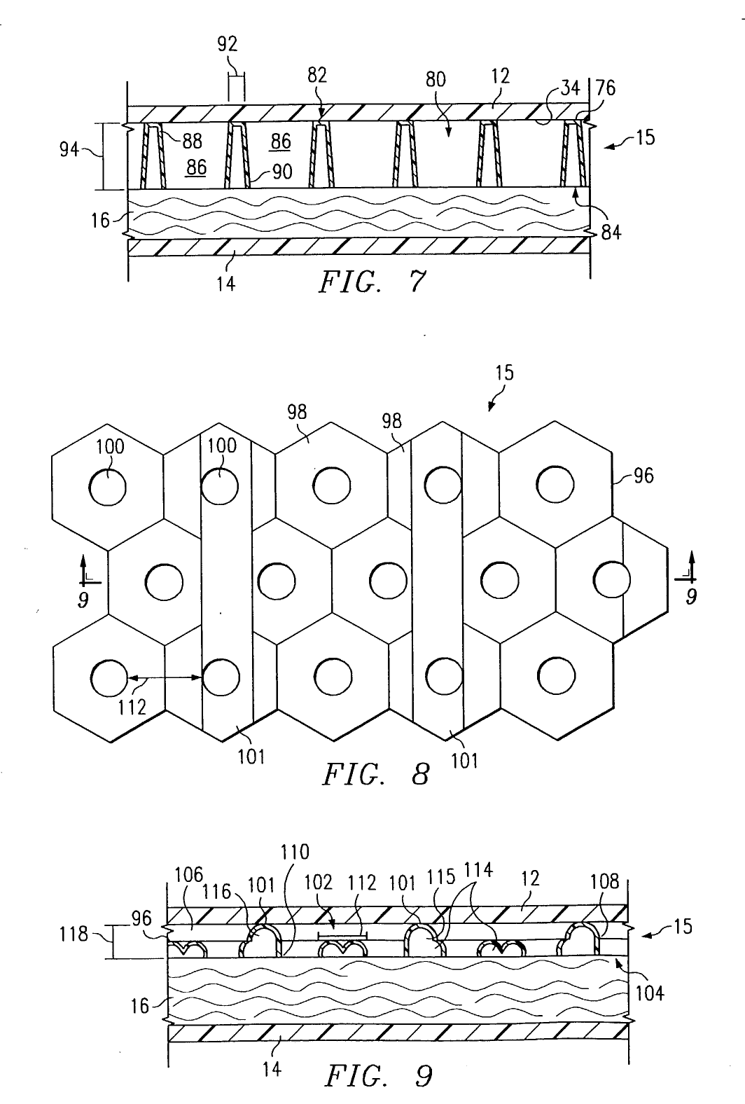

Referring now to FIGS. 6 and 7, a second embodiment of an improved absorbent article of the applicant's invention utilizes an acquisition distribution layer 15 made of a three dimensional apertured film 76 imparted with a hexagonal pattern. Although a hexagonal pattern is discussed herein, it should be understood that other patterns may also be used. Examples of other patterns include circular, oval, elliptical, polygonal, or other suitable patterns or combinations of patterns. The hexagonal pattern forms a plurality of adjacent hexagons or cells 78. In the preferred embodiment, each cell 78 is 1/32″ to ½″ as measured from the flat to flat portion of the hexagon making up each cell 78 of the hexagonal pattern. More preferably, cells 78 of 1/16″ to ⅕″ are used Still more preferably, cells 78 measuring ⅛″ across are used.

Referring more particularly to FIG. 7, which shows an enlarged cross sectional view of film 76 taken along line 7-7 of FIG. 6, three dimensional apertured film 76 has a body facing side or female side 82 and a garment facing side or male side 84. The garment-facing side 34 of the top layer 12 is preferably maintained in close contact with the female side 82 of the apertured plastic film 76. Preferably, top layer 12 maintains contact with but is unbonded to film 76.

As can be seen in FIG. 7, the film 76 is located between a top layer 12 and an absorbent core 16. The apertured plastic film 76 is a three-dimensional structure having a plurality of large openings or buckets 86, each of which has a base opening 88 and an apex opening 90. The apex openings 90 of buckets 86 are in intimate contact with the absorbent core 16, and preferably apex opening 90 is affixed to core 16 to insure this intimate contact. A land area 92 is formed between adjacent apertures 80 on the female side 82 of the apertured plastic film 76. In the honeycomb embodiment, land area 92 is preferably relatively narrow. The three dimensional apertured film 76 has a loft 94 (FIG. 7), i.e. the distance between the surface on the female side 82 and the planar surface on the male side 84, of greater than 30 mils. In the preferred embodiment, the loft 94 is 50 mils.

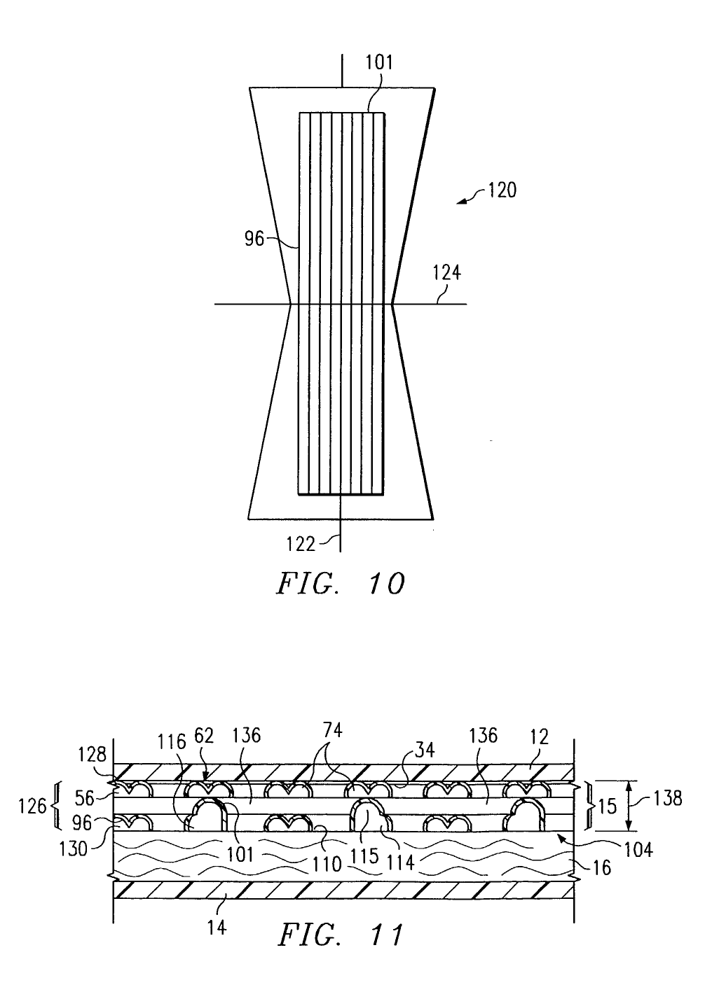

Referring now to FIGS. 8 and 9, a third embodiment of an improved absorbent article of the applicant's invention utilizes an acquisition distribution layer 15 made of a three dimensional apertured film 96 imparted with a hexagonal pattern. Although a hexagonal pattern is discussed for purposes of illustration, it should be understood that other patterns may also be used for any of the films discussed herein. Examples of other patterns include circular, oval, elliptical, polygonal, or other suitable patterns. The hexagonal pattern forms a plurality of adjacent hexagons or cells 98. In the preferred embodiment, the hexagonal pattern is based on a 8.75 mesh wherein “mesh” is the number of cells 98 aligned in a one-inch length. Although a mesh count of 8.75 is preferred, a mesh count of from 2 to 25 or more preferably from 4 to 15 may be used. Preferably, each cell 98 is provided with apertures 100 that have large hole diameters, eg., 59 mils. A plurality of raised ridges 101 are formed on the three dimensional apertured film 96. The raised ridges 101 preferably run longitudinally or parallel to longitudinal centerline 22 (FIG. 1) of the absorbent article 10.

Referring in particular to FIG. 9, which shows an enlarged cross sectional view of film 96 taken along line 9-9 of FIG. 8, three dimensional apertured film 96 has a body facing side or female side 102 and a garment facing side or male side 104. The garment-facing side 34 of the topsheet 12 is preferably maintained in close contact with the female side 102 of the apertured plastic film 96. Preferably, top layer 12 maintains contact with but is unbonded to film 96. The thermoplastic material used in the film 76 preferably has a density in the range of from about 0.919 g/cc to 0.960 g/cc, with the more preferred range of densities being from about 0 930 g/cc to 0.950 g/cc. The general melt indices range for a typical material is preferably from about 0.10 to about 8.50, with the more preferred range typically being from about 1.5 to about 4.5.

As can be seen in FIG. 9, the film 96 is located between topsheet 12 and an absorbent core 16. The apertured plastic film 96 is a three-dimensional structure having a plurality of capillaries 106, each of which has a base opening 108 and an apex opening 110. The apex openings 110 of capillaries 106 are in intimate contact with the absorbent core 16, and preferably apex openings 110 are affixed to core 16 to insure this intimate contact. It should also be noted that essentially only the apex openings 110 of capillaries 106 are in intimate contact with the core 16, thereby assuring that the void spaces 114-116 providing for lateral spillage remain substantially unencumbered. A land area 112 is formed between adjacent apertures 100 on the female side 102 of the apertured plastic film 96. A void volume space 114 is formed on the male side 104 of the apertured plastic film 96 that provides a fluid passageway between each of the cells 98. A channel 115 (FIG. 9) is formed on the male side 104 of each raised ridge 101. An enlarged void volume space 116 is formed when the channel 115 communicates with the void volume space 114 of the apertured plastic film 96. The three dimensional apertured film 96 has a loft 118 (FIG. 9), i.e. the distance between the surface on the raised ridges 101 on female side 102 and the planar surface of the male side 104, in the range of 0.065, i.e., the raised ridge 101 preferably adds 0.015″ to the preferred loft of 0.050″ for film 96. Although 0 050″ is the most preferred loft, a loft of from 0.031″ to 0 125″ and more preferably 0.045″ to 0.100″ may be used. Raised ridges 101 may be formed by affixing a wire around the circumference of a vacuum forming screen or by forming an elongated protrusion upon a vacuum formed screen and passing a film over the screen in a manner known in the art. The thermoplastic material used in the film 96 preferably has a density in the range of from about 0.919 g/cc to 0.960 g/cc, with the more preferred range of densities being from about 0.930 g/cc to 0.950 g/cc. The general melt indices range for a typical material is preferably from about 0.10 to about 8.50, with the more preferred range typically being from about 1.5 to about 4.5.

A disposable diaper 120 utilizing a section of three dimensional apertured film 96 having raised ridges 101 is shown in FIG. 10. Disposable diaper 120 has a longitudinal centerline 122 and a transverse centerline 124. It should be understood that disposable diaper 120 is shown here as an example only, and the invention described herein should not be construed to be limited to disposable diapers but may also include incontinent articles, sanitary napkins, pantiliners or other absorbent articles.

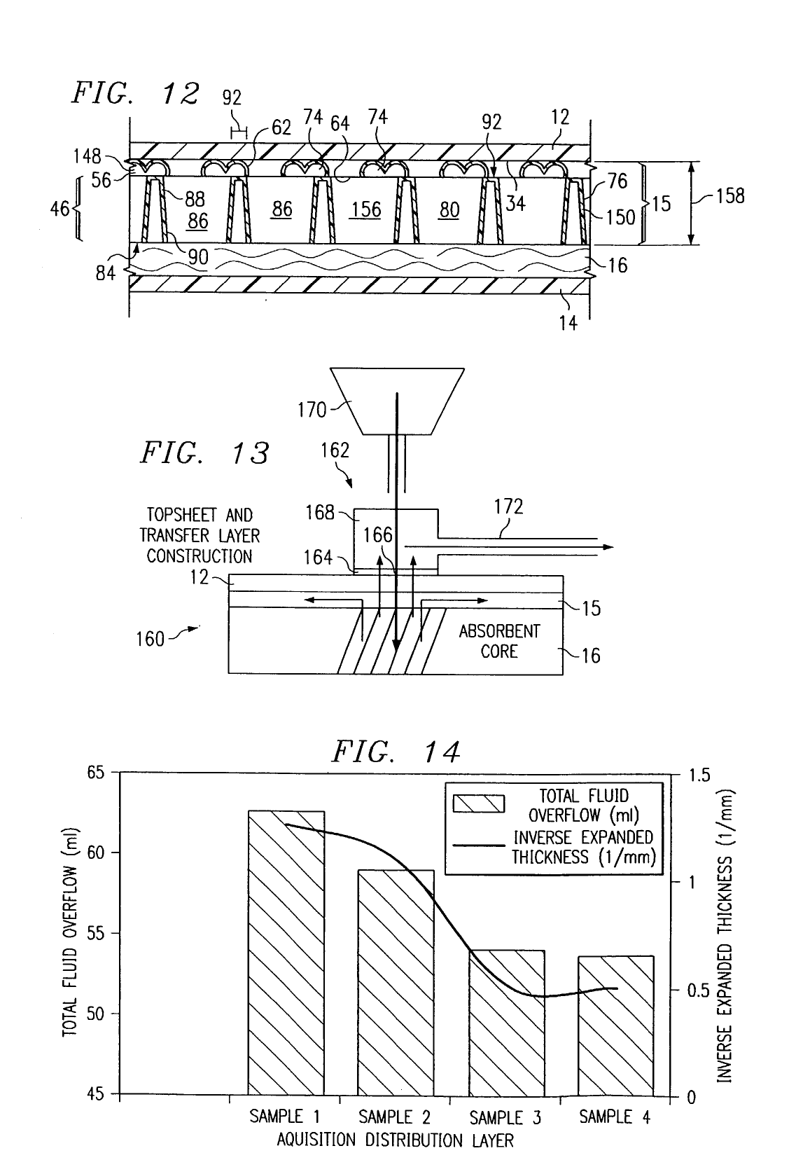

Referring now to FIG. 11, a fourth embodiment of an improved absorbent article of the applicant's invention utilizes an acquisition distribution layer 15 made of three dimensional apertured film 56 (FIGS. 4 and 5) and three dimensional apertured film 96 (FIGS. 8 and 9), which shall be referred to as multi-layer apertured film 126. Three dimensional apertured film 56 forms the body facing sublayer 128 of multi-layer apertured film 126. Three dimensional apertured film 96 forms the garment facing sublayer 130 of multi-layer apertured film 126. The garment-facing side 34 of the topsheet 12 is preferably maintained in close contact with the female side 62 of the apertured plastic film 56 that forms the body facing sublayer 128. Preferably, top layer 12 maintains contact with but is unbonded to sublayer 128.

As can be seen in FIG. 11, the multi-layer apertured film 126 is located between a topsheet 12 and an absorbent core 16. The multi-layer apertured film 126 is a three-dimensional structure that allows fluids to pass therethrough. The three dimensional apertured film 56 that forms the body facing sublayer 128 is in contact with raised ridges 101 that are formed on the sublayer 130. The apex openings 110 of the three dimensional apertured film 96 that forms the garment facing sublayer 130 are preferably in intimate contact with the absorbent core 16. The void volume space 114 and channel 115, which form the enlarged void volume space 116, of the apertured plastic film 96 that forms the garment facing sublayer 130 is complimented by the additional void volume space 74 of three dimensional apertured film 56 that forms the body facing sublayer 128. A further enlarged void volume space 136 is formed by the space between the sublayers 128 and 130 as a result of the height of channels 101. The multi-layer apertured film 126 has a loft 138, i.e the distance between the female side 62 of the three dimensional apertured film 56 that forms the body facing sublayer 128 and the planar surface of the male side 104, of three dimensional apertured film 96. The preferred loft 138 for the multi-layer apertured film 126 is 0.90″, which is the sum of a preferred loft of 50 mils for film 96, 15 mils for raised ridges 101 and 25 mils for top layer 12. Sublayers 128 and 130 of multi-layer film 126 are preferably bonded together in a manner taught by U.S. Pat. No. 5,635,275 to Biagioli, et al., entitled, “Lamination of non-apertured three-dimensional films to apertured three-dimensional films and articles produced therefrom”. U.S. Pat. No. 5,635,275 is hereby incorporated by reference. However, the multi-layer film 126 is preferably unbonded to topsheet 12.

Referring now to FIG. 12, a fifth embodiment of an improved absorbent article of the applicant's invention utilizes an acquisition distribution layer 15 made of three dimensional apertured film 56 (FIGS. 4 and 5) and three dimensional apertured film 76 (FIGS. 6 and 7), which shall be referred to as multi-layer apertured film 146. Three dimensional apertured film 56 forms the body facing sublayer 148 of multi-layer apertured film 146. Three dimensional apertured film 76 forms the garment facing sublayer 150 of multi-layer apertured film 146. The garment-facing side 34 of the topsheet 12 is preferably maintained in close contact with the female side 62 of the apertured plastic film 56 that forms the body facing sublayer 148. Preferably, top layer 12 maintains contact with but is unbonded to sublayer 148.

As can be seen in FIG. 12, the multi-layer apertured film 146 is located between a topsheet 12 and an absorbent core 16. However, it is contemplated that multi-layer apertured film 146 could also function without topsheet 12. The multi-layer apertured film 146 is a three-dimensional structure that allows fluids to pass therethrough. The three dimensional apertured film 56 that forms the body facing sublayer 148 is in contact with land area 92 of three dimensional apertured film 76 that forms the sublayer 150. The body facing sublayer 148 separates the topsheet 12 from unabsorbed fluids that spill over from bucket 86 to an adjacent bucket 86. The void volume space 74 of body facing sublayer 148 and the buckets 86 of garment facing sublayer 150 form a further enlarged void volume space 156. The multi-layer apertured film 146 has a loft 158, i.e the distance between the female side 62 of the three dimensional apertured film 56 that forms the body facing sublayer 148 and the planar surface of the male side 84, of three dimensional apertured film 76. The preferred loft 158 for the multi-layer apertured film 146 is 70 mils, i.e., 50 mils for the garment facing sublayer 150 and 20 mils for the body facing sublayer 148. Sublayers 148 and 150 of multi-layer film 146 are preferably bonded together in a manner taught by U.S. Pat. No. 5,635,275 to Biagioli, et al., which is hereby incorporated by reference. However, the multi-layer, film 146 is preferably unbonded to topsheet 12. The composite multi-layer apertured films 126 and 146 may be constructed in accordance with the teachings of U.S. Pat. No. 5,635,275 to Biagioli, et al., which is hereby incorporated by reference.

In practice, the three dimensional apertured films 56, 76, 96 and multi-layer apertured films 126 and 146 may be used as an acquisition distribution layer 15 in an absorbent article 10. Absorbent article 10 is used for applications where fluid absorption is desirable. In use, body exudates, such as an urine insults from male or female babies or adults, are deposited on the absorbent article 10. The urine insults are typically delivered in a generally singular point of fluid flow. Upon repeated insults, an undesirable leakage or undesirable feeling of wetness by the user may occur due to the core material 16 becoming saturated in the repeat insult region. In other words, the absorbent core 16 may experience an inability to absorb repeated insults in a particular region. As a result, additional fluid insults that are delivered to the absorbent article 10 may be unabsorbed by the core 16 and remain on the top or body facing side 17 of the core layer 16. Applicant's invention provides a method for the unabsorbed fluid from the core layer 16 to be directed to unsaturated zones of the core layer 16. Narrow land areas 92 on the female side 82 of film 76 preferably have a small enough surface area such that fluid contained thereon is insufficient in amount to provide a wetness sensation to the user when portions of the topsheet 12 are momentarily wetted by the spill over of unabsorbed fluid from one bucket 86 to an adjacent bucket 86. When unabsorbed fluid contacts topsheet 12 an unpleasant feeling of wetness of topsheet 12 occurs. A wet topsheet 12 results in uncomfortable fluid contact with the skin of a wearer.

For example, when three dimensional apertured film 56 (FIGS. 4 and 5) is used in absorbent article 10 (FIG. 1), fluid that is not absorbed or that spills-over from core layer 16 is able to flow within void volume space 74 to an unsaturated area of core 16. The void volume space 74 on the male side 64 (FIG. 5) of adjacent cells 58 (FIG. 4) are interconnected to allow a high volume of fluid to pass to unsaturated regions of core 16. The plurality of adjacent hexagons form a large under-side void volume space that provides space for fluid that spills over the top plane or body facing side 17 of saturated core regions 16 and find new, unsaturated regions. The unabsorbed fluid that results from repeated insults may then flow from a saturated zone of absorbent core material 16 and be redirected through the under-side void volume space 74 to an unsaturated zone of the absorbent core material 16. Without the void volume space 74 of the three dimensional apertured film 56, the topsheet 12, which is contact with the skin, will become wet as the insult fluid seeks new regions to be absorbed. The male side void volume area 74 is a much greater total void volume area than previously known anti-rewet or anti-wicking layers.

As another example, when three dimensional apertured film 76 (FIGS. 6 and 7) is used in absorbent, article 10 (FIG. 1), insult fluid that is delivered to an area after core material 16 in the area has been saturated pools within buckets 86. When a bucket 86 at the insult point becomes full, buckets 86 adjacent to the insult point are filled as the fluid within full bucket 86 spills over. This process is repeated as spill-over occurs between adjacent buckets 86 to accommodate the full insult fluid volume. Eventually, the spill-over from buckets 86 flows into a bucket 86 that is located proximate an area of unsaturated core material 16 and the fluid is absorbed. Since the spill over of unabsorbed fluid from a bucket 86 to adjacent buckets 86 disperses the unabsorbed liquid over a larger area of core material 16 where the fluid may be absorbed, an undesirable wetness of the topsheet 12 may be avoided. The open-cell void volume areas 86, is much more total void volume area than previously known film anti-rewet or anti-wicking layers. The preferred percentages range of land areas 92 for three dimensional apertured film 76 is 5 to 20% of the total surface area. The large patterned acquisition distribution layer material or three dimensional apertured film 76 also provides a greater measure of loft, e g greater than 30 mils and more preferably, 50 mils in the ⅛″ honeycomb embodiment. The greater loft 94 or thickness between the upper-most plane and lower-most plane of the of the three dimensional apertured film 64 provides a ‘wick-proof’ barrier or layer between the wetted core 16 and the skin contact area of a user. A greater loft 94 results in an improved feeling of dryness. Since the material in the topsheet 12 is only a small percent of the total occupied volume, the greater the volume, the more “air cushion” that is provided next to the skin contact region.

As a still further example, when three dimensional apertured film 96 (FIGS. 8 and 9) is used in absorbent article 10 (FIG. 1), insult fluid that is not absorbed in core layer 16 is able to flow within void volume space 114. The void volume space 114 on the male side 104 (FIG. 9) of adjacent cells 98 (FIG. 9) are interconnected to allow a high volume of fluid to pass to unsaturated regions of core 16. Additionally, raised ridges 101 form channels 115 to further accommodate unabsorbed fluids via enlarged void volume space 116. A further advantage of the channels 115 is that the channels 115 direct unabsorbed fluids in a desired direction, such as in the longitudinal direction, i e., parallel to longitudinal centerline 122 of disposable diaper 120 (FIG. 10). By directing the unabsorbed fluid in the longitudinal direction, the fluid may be directed to locations with greater amounts of unsaturated core material 16 as opposed to directing the fluid towards undesirable locations such as a perimeter of the diaper. The channels 115 direct fluid away from a direction that is parallel to the transverse centerline of disposable diaper 120. The raised ridges are, therefore, effective at eliminating side leakage from disposable diaper 20.

Additionally, various embodiments of acquisition distribution layer 42 may be combined into a multi-layer apertured film, such as film 126 (FIG. 11) or film 146 (FIG. 12). Multi-layer apertured film 126 provides a further enlarged void volume space 136 to accommodate unabsorbed fluids. The further enlarged void volume space 136 allows unabsorbed fluids to flow to regions where core material 16 is unsaturated without allowing the unabsorbed fluids to come into contact with the topsheet 12, thereby avoiding an unpleasant feeling of wetness for the user.

Multi-layer apertured film 146 (FIG. 12) provides a further enlarged void volume space 156 to accommodate unabsorbed fluids. The further enlarged void volume space 156 allows unabsorbed fluids to spill over lands 92 from buckets 86 to adjacent buckets 86 where core material 16 is unsaturated. Body facing sublayer 148, i.e. film 56, substantially prevents unabsorbed fluids from contacting the topsheet 12 when unabsorbed fluids spill over land 92 from a bucket 86 of garment facing sublayer 150, i.e. film 76, to adjacent buckets, thereby further reducing the unpleasant feeling of wetness for the user.

The use of three dimensional apertured films 56, 76, 96, and multi-layer apertured films 126 and 146 increase the loft of the acquisition distribution layer 15 of the absorbent article 10. The greater loft 75, 94, 118, 138 and 158 or thickness between the upper-most plane and lower-most plane of the of the three dimensional apertured films 56, 76, 96, and multi-layer apertured films 126 and 146 provides a ‘wick-proof’ barrier or layer between the wetted core 16 and the skin contact area of a user. A greater loft 75, 94, 118, 138 and 158 results in an improved feeling of dryness. Since the material in the topsheet 12 is only a small percent of the total occupied volume, the greater the volume, the more “air cushion” that is provided next to the skin contact region.

The large female side void volume of the “spill-over” embodiments facilitates dispersion of unabsorbed fluids. Preferably, for a square meter of film, the female side void volume is greater than 500 cm³, more preferably greater than 750 cm³, and most preferably greater than 1000 cm³. Additionally, the large male side void volume of the “spill-under” embodiments also facilitates dispersion of unabsorbed fluids. Preferably, for a square meter of film, the male side void volume is preferably greater than 500 cm³, more preferably greater than 600 cm³, and most preferably greater than 750 cm³.

Test Methods and Data

Acquisition Distribution Layer Testing

Testing was performed using the Multiple Insult Acquisition method Several methods are described in detail in an article by James P Hanson in an article appearing in Nonwovens World, Fall 1997, page 57-63, entitled, “The Test Mess Part III—Credible Testing for Liquid Acquisition”, which is incorporated herein by reference.

More specifically, the applicant's test was conducted as follows. Referring now to FIG. 13, die cut samples 160 are cut from absorbent article 10 in an area where acquisition distribution layer 15 is present. The topsheet 12 and acquisition distribution layer 15 are removed from the absorbent article 10, paying particular attention not to change the orientation of the materials. The topsheet 12 and acquisition distribution layer 15 of the core cuts or die samples 160 are then randomly weighed and the average weight and standard deviation for the weight are randomly recorded. Each die cut sample 160 is then reconstructed by adding the absorbent core 16.

To perform the Acquisition Rate Performance on all three layers, a Liquid Acquisition Apparatus 162 is used. Apparatus 162 is made up of a plate 164 having an opening 166 in the center of the plate 164 for placement on top of sample 160. A controlled volume chamber 168 extends upwardly from the plate 164 for receiving a desired fluid flow rate and dosage from a fluid supply 170. An overflow pipe 172 extends outwardly from the controlled volume chamber 168 at a location slightly above the plate 164.

Six samples were tested by the above described method wherein the fluid supply 170 pumped fluid into the controlled volume chamber 168 at a rate of 7 ml/sec. Samples 1-3 are samples having an acquisition distribution layer similar to that shown in FIG. 3 wherein the samples have varying amounts of loft or thickness as is indicated in Table 1, below. In particular, Sample 1 is a prior art film in accordance with the teachings of United States Invention Registration no H1670, to Aziz et al. having 20 mils of loft, a pattern of round or hex cells and a 22 mesh count. Sample 2 is a prior art film in accordance with the teachings of United States Invention Registration no. H1670, to Aziz et al. having 23 mils of loft, a pattern of hex cells and a 25 mesh count. Samples 4 and 5 are examples of films embodying the invention of the application wherein Sample 3 has an acquisition distribution layer 15 with male side void volume flow area similar to that shown in FIGS. 4 and 5. Sample 4 is the embodiment of the invention shown in FIGS. 6 and 7, i.e., the “bucket” embodiment, having a ⅛″ honeycomb pattern on the acquisition distribution layer. Sample 3 has slightly lower loft (it is 49 mils vs. 51 mils) but a greater male side void volume than Sample 4. In particular, Sample 3 has a hex pattern with 50 mils loft on a 8.75 mesh count. Sample 4 has a ⅛″ honeycomb pattern with 50 mils of loft on an 8 mesh count. The results are shown in Table 1, below.

| | | Inverse | | |
|---|---|---|---|---|
| | Total Fluid | Expanded Loft | | |
| Sample No. | Overflow (ml) | (1/mm) | Loft (mm) | Mesh |
| Sample 1 | 62.71 | 0.787402 | 20 | 22 |
| Sample 2 | 59.09 | 0.905512 | 23 | 25 |
| Sample 3 | 54.15 | 1.929134 | 49 | 8.75 |
| Sample 4 | 52.65 | 2.007874 | 51 | 8 |

The results of the test is shown graphically in FIG. 14. FIG. 14 is comprised of a bar graph that shows fluid overflow (ml) for each sample 1-4. Additionally, FIG. 14 is comprised of a line graph that shows the inverse of the expanded thickness or loft of each sample. Overflow is defined as fluid that flows out of overflow pipe 172 of the Liquid Acquisition Apparatus 162 when 15 mL amount of fluid is delivered at 7 ml/sec into controlled volume chamber 168 The fluid that does not flow through overflow pipe 172 is absorbed by the sample 160.

It can be seen from FIG. 14, that the greater the loft for a particular sample, the less fluid overflow that is observed for a particular sample. The films of applicant's invention, i.e. Samples 3 and 4 have a markedly greater loft than the films having the prior art design, i.e. Samples 1 and 2. Samples 3 and 4 show a markedly lower amount of fluid overflow. It should be noted that the total void volume for Samples 1 and 2 is less than 550 cc/m² of sample material while the total void volume for Samples 3 and 4, which illustrate embodiments of applicant's invention, is more than 1000 cc/m². The preferred total void volume for applicant's invention is greater than 750 cc/m², more preferably greater than 875 cc/m², and most preferably greater than 1000 cc/m².

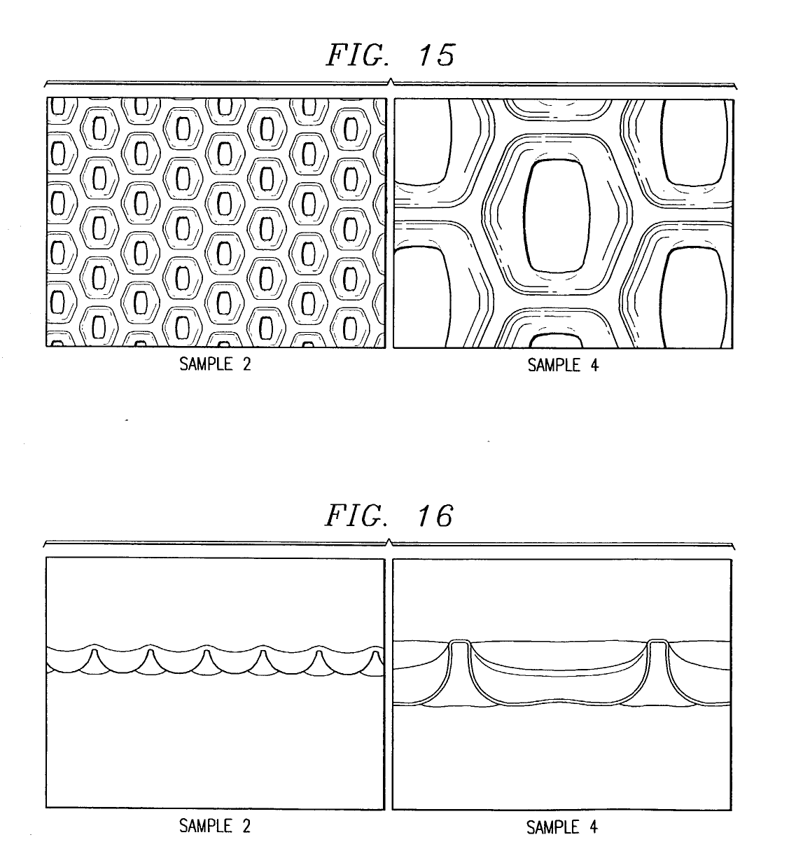

To further illustrate the substantial increase in void volume space of the films of the invention over existing films, microphotographs of Sample 2 and Sample 4 are set forth in FIGS. 15 and 16. FIGS. 15 and 16 show Samples 2 and 4 at 50× magnification. FIG. 15 shows a plan view of samples 2 and 4. FIG. 16 shows a side cross-sectional view of samples 2 and 4. The substantial increase in void volume space is apparent from each of FIGS. 15 and 16.

Finally, microphotographs of known magnification and scale of dimensions were taken of each of Samples 1-4 to enable empirical calculations of void volume spaces. While the cells of the embodiments described herein are best approximated as a geometric frustum, as taught in Thompson U.S. Pat. No. 4,939,135, it is within the scope of the invention to include other cell shapes such as substantially straight walled cells, as taught in Radel U.S. Pat. No. 4,342,314, and cells which converge to a narrow point and then diverge again toward the apertured end, as taught by Rose U.S. Pat. No. 4,895,749. The resulting geometric calculation for void volume space data for Female Side void volume, Male Side void volume, and the Total Void Volumes are shown below in Table 3.

TABLE 3

| | | | | Female | Male | Total |
|---|---|---|---|---|---|---|
| | | | | side | side | Void |
| Sample | Loft | | Cells/m² of | volume | volume | Volume |
| No | (mils) | Mesh | film | (cc/m²) | (cc/m²) | (cc/m²) |
| 1 | 20 | 22 | 872,170 | 189 | 339 | 528 |
| 2 | 23 | 25 | 1,090,755 | 247 | 236 | 483 |
| 3 | 49 | 8.75 | 131,771 | 294 | 752 | 1046 |
| 4 | 51 | 8 | 105,649 | 1357 | 475 | 1832 |

It can be seen from table 3 that the “spill-under” embodiment of applicant's invention, demonstrated by Sample 3 has a substantially greater male side void volume, i.e., 752 cc/m², than do any of the other samples. The “spill-over” embodiment of applicant's invention, demonstrated by Sample 4 has a substantially greater female side volume, i.e. 1357 cc/M², than do any of the other samples.

From the above, it will be appreciated that applicant's invention will reduce or eliminate the wetness sensation felt by the user during and after repeated insults as unabsorbed fluid flows from an area of saturated core material to an area of unsaturated core material for absorption. Applicant's invention redirects unabsorbed fluids to non-saturated areas of a core material 16 while preventing substantial contact of the unabsorbed fluids with the topsheet 12. The invention of the applicant prevents an unpleasant feeling of wetness of the topsheet 12 while providing the ability to receive multiple insults at a singular point.

Finished Product Test

It is well known in the art that finished absorbent articles vary in construction and have differing performance characteristics Accordingly, applicant discloses the finished product testing methods employed to compare finished absorbent articles having no incorporated ADL to finished absorbent articles having an incorporated ADL. Tests to quantify a nonwoven material's performance have been developed and are widely accepted by the industry. Two of these tests, established by the European Disposables and Nonwovens Association, are EDANA 150.4-99 and EDANA 151.2-99. These tests result in the quantification of liquid strike-through time and wetback, respectively Both tests were designed to evaluate the performance of nonwoven layers themselves rather than the liquid strike-through time and wetback of finished absorbent articles. However, the principles behind the EDANA testing methods are found to be directly applicable when used to evaluate the effects of incorporating an ADL into a finished absorbent article. The EDANA test methods have been adapted to provide the finished product test. This test offers a consistent means for quantifying a finished absorbent article's finished product liquid strike-through time and finished product wetback. Once these values are obtained, finished absorbent articles with an incorporated ADL and finished absorbent articles with no incorporated ADL can be compared to determine which finished absorbent articles are superior with regard to finished product liquid strike-through time and finished product wetback. Generally, a product with lower finished product liquid strike-through time and lower finished product wetback is superior to a product with higher finished product liquid strike-through time and higher finished product wetback since the lower values represent faster absorption and a drier article after insults, respectively.

Generally, the finished product test consists of procedural steps which include insulting finished absorbent products with simulated urine, measuring finished product liquid strike-through time, allowing some period of time to elapse while the urine is absorbed by the absorbent core of the finished absorbent article, measuring finished product wetback, and repeating the steps until three insults have been administered and three measurements of finished product liquid strike-through time and finished product wetback have been recorded.

All steps of the finished product test are performed in a climate controlled room having a temperature of 23° C. and a relative humidity of 50%.

The simulated urine is produced by Lab Chem Inc., supplied by Fisher Scientific of Hanover Park, Ill. Furthermore, the simulated urine is ACS grade, having pH between 6.7 and 7.3 to achieve a surface tension of 70 dyne, +/−5.

The filter paper should measure 4″×4″, have a mass per unit area of approximately 140 g/m². Such filter paper is produced by Ahlmstrom as model topsheet core paper grade 989 and supplied by Empirical Manufacturing Company Inc. of Cincinnati, Ohio.

Once the filter paper and the samples have been acclimated to the climate control room as described above, the next step of the test procedure may be performed. The next step is performed by unfolding a sample and taping the sample to a level nonabsorbent table. The sample is taped and secured so that the topsheet is facing up and the edges of the sample lie generally parallel to the table, if not actually affixed to the table with tape. The samples are manipulated such that any leg elastics, a common feature of finished absorbent articles, are taut and remain upright to prevent fluid from running off of the sample onto the table.

The front edge of a sample is identified as the edge that would be most closely positioned to the user's stomach during proper use. The back edge is identified as the edge that would be most closely positioned to the user's back during proper use. The insult point for a baby diaper is a point which is located by viewing the sample from above and measuring 6″ from the front edge of the sample's absorbent core toward the back edge of the sample's absorbent core and along the centerline of the absorbent core's narrower dimension. Similarly, the insult point for adult incontinent briefs is a point which is located by viewing the sample from above and measuring 13″ from the front edge of the sample's absorbent core toward the back edge of the sample's absorbent core and along the centerline of the absorbent core's narrower dimension. Insult points may be identified and marked before or after the samples have been affixed to the table.

Further test preparation includes filling three graduated cylinders with simulated urine until the appropriate volume in each cylinder has been reached. The appropriate volume for testing a baby diaper sample is 80 ml while the appropriate volume for testing an adult incontinent brief is 160 ml.

Also, six stacks of filter paper are created. Each of the stacks contains 10 individual sheets of filter paper. Within fifteen minutes of insulting the samples, the stacks are each weighed and their respective weights are recorded on the top sheet of filter paper of each stack.

The test is continued by aligning the central axis of a plastic cylinder with the sample's insult point, ensuring that the cylinder's central axis is orthogonal to the plane of the table. Then the cylinder is lowered toward the table until it contacts the sample in a manner that creates a seal with the sample. The seal should be adequate to later prevent simulated urine from passing from the cylinder without exiting the cylinder through the topsheet of the sample. However, no greater downward force should be applied to the cylinder than is necessary to achieve the above described seal. The plastic cylinder has an inside diameter of 51 mm and a height of 100 mm. Such cylinders are available from Fisher Scientific as catalog number 08-570-21D, Fisher PP Cylinder 100 ml capacity, 1 ml subdivisions.

While maintaining the above described seal between the plastic cylinder and the sample, the first insult is performed. This is accomplished by pouring the predetermined volume of simulated urine from the graduated cylinder into the plastic cylinder. It should be noted that the graduated cylinder is not positioned a great distance above the top edge of the plastic cylinder but rather at a level very near the top edge of the plastic cylinder. The predetermined volume of simulated urine is fully poured into the plastic cylinder without delay. As soon as the simulated urine contacts the topsheet of the sample, a chronograph is operated to begin calculating the elapsed time. When the liquid has fully exited the bottom of the plastic cylinder through the topsheet of the sample, the chronograph is stopped and the elapsed time is recorded as the finished product liquid strike-through time for insult ‘A’.

The plastic cylinder is removed from the sample and the chronograph is stopped, reset to zero, and restarted to allow for a new calculation of elapsed time. After ten minutes has passed, the stopwatch is again stopped and reset to zero.

Immediately after the ten minutes has passed, one of the pre-weighed stacks of dry filter paper is examined and its weight is recorded on a data sheet as dry filter paper for insult ‘A’, the stack is centered above the insult point and lowered gently downward until it contacts the sample. Immediately following the placement of the filter paper, an 8 lb weight is aligned with the filter papers in a manner that the square bottom surface of the weight covers the entire area of the square filter papers below it. The weight is gently lowered onto the filter paper. Such weights are known as 816 strike through assemblies supplied by Concord-Rem Company of Cincinnati, Ohio. as Model RDL-0833-C. The weight's bottom surface is covered by a polyethylene covered foam having a hydrophobic surface. When the weight has been placed on the filter paper, the chronograph is once again started.

After two minutes have passed since the weight was placed on the filter paper, the weight is removed from the filter paper and the stack of filter paper is removed from the sample. The stack of filter paper is immediately weighed and the weight of the filter paper is recorded on a data sheet as wet filter paper for insult ‘A’.

The test is continued by aligning the central axis of a plastic cylinder with the sample's insult point, ensuring that the cylinder's central axis is orthogonal to the plane of the table. Then the cylinder is lowered toward the table until it contacts the sample in a manner that creates a seal with the sample. The seal should be adequate to later prevent simulated urine from passing from the cylinder without exiting the cylinder through the topsheet of the sample. However, no greater downward force should be applied to the cylinder than is necessary to achieve the above described seal. The plastic cylinder is the same as used previously.

While maintaining the above described seal between the plastic cylinder and the sample, the second insult is performed. This is accomplished by pouring the predetermined volume of simulated urine from the graduated cylinder into the plastic cylinder. It should be noted that the graduated cylinder is not positioned a great distance above the top edge of the plastic cylinder but rather at a level very near the top edge of the plastic cylinder. The predetermined volume of simulated urine is fully poured into the plastic cylinder without delay. As soon as the simulated urine contacts the topsheet of the sample, a chronograph is operated to begin calculating the elapsed time. When the liquid has fully exited the bottom of the plastic cylinder through the topsheet of the sample, the chronograph is stopped and the elapsed time is recorded as the finished product liquid strike-through time for insult ‘B’.

The plastic cylinder is removed from the sample and the chronograph is stopped, reset to zero, and restarted to allow for a new calculation of elapsed time. After ten minutes has passed, the stopwatch is again stopped and reset to zero.

Immediately after the ten minutes has passed, two of the pre-weighed stacks of dry filter paper are combined, their combined weight recorded on a data sheet as dry filter paper for insult ‘B’, the stack is centered above the insult point and lowered gently downward until the stack contacts the sample. Immediately following the placement of the filter paper, the 8 lb weight is aligned with the filter papers in a manner that the square bottom surface of the weight covers the entire area of the square filter papers below it. The weight is gently lowered at onto the filter paper. When the weight has been placed on the filter paper, the chronograph is once again started.

After two minutes have passed since the weight was placed on the filter paper, the weight is removed from the filter paper and the stack of filter paper is removed from the sample. The stack of filter paper is immediately weighed and the weight of the filter paper is recorded as wet filter paper for insult ‘B’.

The test is continued by aligning the central axis of a plastic cylinder with the sample's insult point, ensuring that the cylinder's central axis is orthogonal to the plane of the table. Then the cylinder is lowered toward the table until it contacts the sample in a manner that creates a seal with the sample. The seal should be adequate to later prevent simulated urine from passing from the cylinder without exiting the cylinder through the topsheet of the sample. However, no greater downward force should be applied to the cylinder than is necessary to achieve the above described seal. The plastic cylinder is the same as used previously.

While maintaining the above described seal between the plastic cylinder and the sample, the third insult is performed. This is accomplished by pouring the predetermined volume of simulated urine from the graduated cylinder into the plastic cylinder. It should be noted that the graduated cylinder is not positioned a great distance above the top edge of the plastic cylinder but rather at a level very near the top edge of the plastic cylinder. The predetermined volume of simulated urine is fully poured into the plastic cylinder without delay. As soon as the simulated urine contacts the topsheet of the sample, a chronograph is operated to begin calculating the elapsed time. When the liquid has fully exited the bottom of the plastic cylinder through the topsheet of the sample, the chronograph is stopped and the elapsed time is recorded as the finished product liquid strike-through time for insult ‘C’.

The plastic cylinder is removed from the sample and the chronograph is reset to zero and restarted to allow for a new calculation of elapsed time. After ten minutes has passed, the stopwatch is again stopped and reset to zero.

Immediately after the ten minutes has passed, three of the pre-weighed stacks of dry filter paper are combined, their combined weight recorded on a data sheet as dry filter paper for insult ‘C’, the stack is centered above the insult point and lowered gently downward until the stack contacts the sample. Immediately following the placement of the filter paper, the 8 lb weight is aligned with the filter papers in a manner that the square bottom surface of the weight covers the entire area of the square filter papers below it. The weight is gently lowered onto the filter papers. When the weight has been placed on the filter paper, the chronograph is once again started.

After two minutes have passed since the weight was placed on the filter paper, the weight is removed from the filter paper and the stack of filter paper is removed from the sample. The stack of filter paper is immediately weighed and the weight of the filter paper is recorded as wet filter paper for insult ‘C’.

The finished product rewet for each insult is later calculated as the difference of the wet stack of filter paper weight for a particular insult and the dry stack of filter paper weight for that particular insult. The rewet value essentially represents the weight of the liquid absorbed by the stack of filter paper during the two minutes it was in contact with the sample.

The above described steps result in the generation of two significant data values for each insult, a finished product liquid strike-through time and a finished product rewet.

The above described finished product test was used to generate finished product liquid strike-through times and finished product rewet values for different samples. In order to demonstrate the benefits of incorporating applicant's ADL within a finished product, samples were chosen in a manner such that for each conventional sample tested, an accompanying sample with applicant's ADL replacing a nonwoven ADL was tested. Substitution of the nonwoven ADL with applicant's ADL is the only substantial difference in the samples which are later compared in Tables 101 through 103. The sample test data is first divided into three tables. Each of these tables display the data generated by testing samples from three different batches. Table 101 contains data for samples manufactured at Curt Joa Converter 1. Similarly, Table 102 contains data for samples manufactured at Lambi Converter 2 and Table 103 contains data for adult incontinence articles.

Furthermore, the samples within the tables are organized in a fashion that provides easy comparison of samples whose only difference in construction is the replacement of a conventional ADL by applicant's ADL. For example, by inspecting Table 101, one can see that sample 11 and sample 14 are being compared because they have similar core construction. Only samples manufactured at the same facility and having the same core construction can be compared to determine which products are tested to have superior finished product liquid strike-through time and finished product rewet.

For example, consider the comparison that can be made between sample 11 and sample 14. First, note that both samples were manufactured at Converter 1 and that they both have the same core construction, 15 grams of pulp material and 8 grams of SAP (super absorbent polymer). Second, note that sample 11 is a conventional finished absorbent article which incorporates a nonwoven sheet as its ADL while sample 14 incorporates applicant's ADL. More specifically, sample 11 contains a 48 gsm resin nonwoven ADL, a 175 mm long core, and a layer of tissue paper both on the top and bottom of the core. Sample 14 has a 175 mm long core, a layer of tissue paper both on the top and bottom of the core, but instead of the resin nonwoven ADL, it contains a Tredegar Film Products (noted in the ADL column of the tables as T) ADL. This particular sample incorporates a Tredegar Film Products 60HX-28079 ADL. It is clear from the data provided in Table 101 for samples 11 and 14 that sample 14 has slightly higher finished product liquid strike-through times. However, it is also apparent that the finished product rewet for sample 14, the sample having applicant's ADL, is significantly reduced during insult B and insult C.

Another example of lowered finished product rewet values for samples containing applicant's ADL is shown by the data for sample 18. Samples 15 and 18 both have a reduced mass of pulp within their cores in comparison to samples 11 and 14. Samples 15 and 18 have 13 grams of pulp instead of the 15 grams of pulp found in samples 11 and 14. Both samples 15 and 18 also have a layer of tissue paper on the top and bottom of the core. Even with the reduced pulp content of the core, the test results for sample 18 show that for the second and third insults, the finished product rewet is again significantly reduced.

TABLE 101
Converter 1 Samples: Size 3 Baby Diapers

| | | | | | | Finished Product | | |
|---|---|---|---|---|---|---|---|---|
| | | | Finished Product Liquid | | | Rewet (g) | | |
| Sample Description | | | Strike-through Time (s) | | | | Insult | Insult |
| ID | ADL | Core | Insult A | Insult B | Insult C | Insult A | B | C |
| 11 | NW | 15Pulp/ | 4.9 | 6.4 | 7.7 | 0.28 | 21.88 | 32.21 |
| 14 | T | 8SAP | 6.5 | 8.0 | 13.5 | 0.28 | 12.60 | 21.06 |
| 15 | NW | 13Pulp/ | 4.9 | 6.4 | 7.9 | 020 | 25.20 | 40.02 |
| 18 | T | 8SAP | 7.5 | 8.0 | 11.1 | 0.24 | 11.18 | 17.44 |

The general trend of lowering finished product rewet values in the second and third insults of samples containing applicant's ADL holds throughout each of the listed test comparisons. Accordingly, it should be noted that sample 1E is shown in Table 102 to have significantly lower finished product rewet values for insults B and C than its conventional counter part, sample 1A Likewise, sample 2E is shown to have significantly lower finished product rewet than its conventional counterpart 2A

TABLE 102
Converter 2 Samples: Size 3 Baby Diapers

| | | | | | | Finished Product | | |
|---|---|---|---|---|---|---|---|---|
| | | | Finished Product Liquid | | | Rewet (g) | | |
| Sample Description | | | Strike-through Time (s) | | | | Insult | Insult |
| ID | ADL | Core | Insult A | Insult B | Insult C | Insult A | B | C |
| 1A | NW | 15Pulp/ | 11.20 | 20.51 | 21.32 | 0.07 | 16.93 | 37.00 |
| 1E | T | 10SAP | 13.79 | 17.44 | 29.01 | 0.08 | 0.92 | 10.84 |
| 2A | NW | 15Pulp/ | 12.73 | 23.94 | 27.06 | 0.07 | 18.49 | 38.44 |
| 2E | T | 8SAP | 12.93 | 16.56 | 23.93 | 0.13 | 3.20 | 12.04 |

When the nonwoven ADL of the large adult briefs were replaced with the applicant's ADL, the finished product liquid strike-through time was reduced as well as the finished product rewet values for the second and third insults Samples 1 and 3 are both large adult briefs that have nonwoven topsheets and the same absorbent core. The difference between sample 1 and sample 3 is that instead of having a nonwoven ADL, sample 3 has an ADL produced by the applicant Sample 3 clearly has lower finished product strike-through times and finished product rewet than sample 1 for each and every insult.

Much like the results of samples 1 and 3, 10 and 8 are also large adult briefs having the same absorbent core but instead of having a nonwoven topsheet, the topsheet of samples 10 and 8 are topsheets produced by applicant. The topsheet is called Soft Edge. As with the other examples, when the conventional ADL of sample 10 is replaced by applicant's ADL, improved finished product liquid strike-through time and finished product rewet were the result. The only exception to that improvement is the slightly higher finished product rewet of sample 8 for insult A.

TABLE 103
Large Adult Briefs

| | | | Finished Product | | | Finished Product | | |
|---|---|---|---|---|---|---|---|---|
| Sample | | | Liquid Strike- | | | Rewet (g) | | |
| Description | | | through Time (s) | | | Insult | Insult | Insult |
| ID | ADL | Topsheet | Insult A | Insult B | Insult C | A | B | C |
| 1 | NW | NW | 25.65 | 41.62 | 49.38 | 0.14 | 28.18 | 42.46 |
| 3 | T | | 21.85 | 22.31 | 28.05 | 0.13 | 2.52 | 14.53 |
| 10 | NW | T | 21.49 | 23.20 | 27.60 | 0.08 | 0.10 | 9.27 |
| 8 | T | | 16.98 | 20.28 | 20.55 | 0.10 | 0.09 | 0.62 |

The present invention is illustrated herein by example, and various modifications may be made by a person of ordinary skill in the art. For example, various geometries, materials and multiple-layer film combinations fall within the scope of the invention. As another example, although the present invention has been described in connection with diapers, incontinent articles, sanitary napkins, and related products, the absorbent articles of the present invention are fully applicable to other, similar products, including, without limitation, other body coverings where absorbent materials may be desired. Such body coverings may include medical drapes, medical gowns, medical smocks, ostomy appliances, feminine hygiene products, body transfer sheets, fluid collection pouches, industrial clean room garments and other products.

It is therefore believed that the present invention will be apparent from the foregoing description. While the methods and articles shown or described have been characterized as being preferred it should be obvious that various changes and modifications may be made therefrom without departing from the spirit and scope of the invention as defined in the following claims.

1. An absorbent article comprising
a topsheet;
a high void volume acquisition distribution layer adjacent the topsheet; and
a reduced absorbent core adjacent the high void volume acquisition distribution layer.

2. The absorbent article of claim 1, wherein the reduced absorbent core is comprised of a combination of pulp and super absorbent polymer and contains 20% less super absorbent polymer than a standard absorbent article.

3. The absorbent article of claim 1, wherein the reduced absorbent core is comprised of a combination of pulp and super absorbent polymer and contains 20% less pulp than a standard absorbent article.

4. The absorbent article of claim 1, wherein the reduced absorbent core is comprised of a combination of pulp and super absorbent polymer and contains 20% less pulp and 20% less super absorbent polymer than a standard absorbent article.

5. The absorbent article of claim 1, wherein the reduced absorbent core comprises 20% less super absorbent polymer than a standard absorbent article.

6. The absorbent article of claim 1, wherein the reduced absorbent core comprises 20% less pulp than a standard absorbent article.

7. The absorbent article of claim 1 wherein the reduced core comprises 20% less pulp and 20% less super absorbent polymer than a standard absorbent article.

8. A method for constructing an absorbent article with a topsheet and an absorbent core, the method comprising the steps
determine the desired strike through and rewet characteristics for the absorbent article;
determine the amount of pulp and super absorbent polymer required to achieve the desired strikethrough and rewet characteristics; reduce the amount of pulp or super absorbent polymer; and
assemble the absorbent article with a high volume acquisition distribution layer between the topsheet and the core.

9. The method of claim 8, wherein the amount of super absorbent polymer is reduced by 20%.

10. The method of claim 8, wherein the amount of pulp is reduced by 20%.

11. The method of claim 8, wherein the amount of pulp is reduced by 20% and the super absorbent polymer is reduced by 20%.

12. An absorbent article comprising:
a topsheet;
a high void volume acquisition distribution layer having a thickness; and
an absorbent core that is reduced in thickness by an amount greater than the thickness of the acquisition distribution layer.

13. A method of doing business comprising the steps:
establish strikethrough and rewet design criteria,
designing an absorbent article having a topsheet and an absorbent core of absorbent material sufficient to achieve strike through and rewet characteristics;
adding a high void volume acquisition distribution layer to the absorbent article;
reducing the amount of absorbent core material in the absorbent article by an amount sufficient to reduce strikethrough or rewet characteristics to about the original design criteria with the added ADL.
